(19) United States
(12) Patent Application Publication
(10) Pub. No.: US 2026/0327171 A1
(43) Pub. Date: Oct. 22, 2026

(54) SEPARATION OF SYNTHESIZED METHANOL

(71) Applicant: L'Air Liquide Société Anonyme pour I'Etude et I'Exploitation des Procédés Georges Claude, Paris (FR)

(72) Inventors: Tobias OELMANN, Bad Vilbel (DE); Michael STROZYK, Offenbach (DE); Vincent PENA, Frankfurt (DE)

(21) Appl. No.: 19/486,703

(22) PCT Filed: May 24, 2024
(86) PCT No.: PCT/EP2024/064440

§ 371 Date: Nov. 21, 2025

(30) Foreign Application Priority Data
Jun. 7, 2023 (EP) 23178025.5

(51) Int. Cl. C07C 29/74

(52) U.S. Cl. CPC C07C 29/74

(57) ABSTRACT

A process for synthesis of methanol with a synthesis reactor assembly, wherein methanol produced with the synthesis reactor assembly is separated with a separation unit from an outlet stream of the synthesis reactor assembly, wherein the separation unit has a plurality of separation means fluidically connected in parallel, wherein the outlet stream is divided between the separation means such that the entire outlet stream is guided through one of the separation means or a portion of the outlet stream is guided through each of a plurality of the separation means, and wherein a division of the outlet stream between the separation means is adjusted in dependence on a volumetric flow rate of the outlet stream.

### CROSS REFERENCE TO RELATED APPLICATIONS

This application is a 371 of International Application No. PCT/EP2024/064440, filed May 24, 2024, which claims priority to European Patent Application No. 23178025.5, filed Jun. 7, 2023, the entire contents of which are incorporated herein by reference.

### BACKGROUND

The invention relates to a process and an arrangement for synthesis of methanol. In this context, the invention is directed in particular to the separation of methanol from an outlet stream of the synthesis reactor assembly that is used.

One of the reactants of the methanol synthesis is hydrogen. In order to achieve a methanol synthesis that is as environmentally friendly as possible it is desirable to use hydrogen from a water electrolysis operated with renewable energy. However, renewable energies fluctuate and so the availability of hydrogen from a corresponding source also fluctuates. An outlet stream of the synthesis reactor assembly also fluctuates with the availability of the reactant hydrogen.

The methanol produced is contained in the outlet stream and is usually separated from the outlet stream with a separator. However, known separators function only within a comparatively limited range of flow rates of the outlet stream. The effective use of a separator requires a flow velocity of the outlet stream entering the separator to be within a defined value range in order that the condensate droplets can be trapped in the separator. If the outlet stream is outside a suitable range, the separation capacity of the separator falls and the risk that liquid droplets will pass through the separator increases. This can damage a downstream compressor.

The separation of the methanol is thus difficult when the outlet stream of the synthesis reactor assembly fluctuates, as is the case in particular with methanol production based on hydrogen which has been produced with renewable energies.

### SUMMARY

The object of the present invention is to make it possible to reliably separate methanol from the outlet stream even in the case of a reactor outlet stream that fluctuates owing to a fluctuating reactant supply.

This object is achieved with the process and the arrangement according to the independent claims. Further advantageous embodiments are specified in the dependent claims. The features presented in the claims and in the description may be combined with one another in any technologically advantageous way.

According to the invention, a process for synthesis of methanol with a synthesis reactor assembly is presented, wherein methanol produced with the synthesis reactor assembly is separated with a separation unit from an outlet stream of the synthesis reactor assembly, wherein the separation unit has a plurality of separation means fluidically connected in parallel, wherein the outlet stream is divided between the separation means such that the entire outlet stream is guided through one of the separation means or a portion of the outlet stream is guided through each of a plurality of the separation means, and wherein a division of the outlet stream between the separation means is adjusted in dependence on a volumetric flow rate of the outlet stream.

With the described process, methanol can be produced from reactants such as hydrogen on the one hand and carbon dioxide and/or carbon monoxide on the other hand. This can take place with the synthesis reactor assembly in a manner known per se. The reactants can be supplied to the synthesis reactor assembly from a raw gas source by way of a feed gas stream. It is not important for the described process whether the reactants are supplied to the synthesis reactor assembly individually or in the form of a mixture. Preferably, one of the reactants is hydrogen which is obtained from an electrolysis operated with renewable energy.

The products of the reaction that takes place in the synthesis reactor assembly are discharged from the synthesis reactor assembly together with the unreacted reactants in the form of an outlet stream. The outlet stream thus contains the methanol which has been produced. In order to make this methanol accessible for further processing, the methanol is separated from the outlet stream. This is carried out with a separation unit. The separation unit can in particular be adapted to separate a liquid and a gas from one another. In this case, the separation unit serves to separate a condensate from a two-phase gas-liquid stream.

In this respect, the separation unit has the same function as a separator which is usually used in a conventional arrangement for synthesis of methanol. The separation unit preferably has a pre-separator and a main separator. The pre-separator and the main separator are preferably flowed through in succession vertically from bottom to top, so that separated condensate flows by gravity to the bottom, where the raw methanol at the bottom of the separation vessel is collected and is then discharged.

The separation unit used in the described process differs in its construction from a conventional separator. In particular, the separation unit has a plurality of separation means fluidically connected in parallel. Fluidically connected in parallel means that a specific portion of the outlet stream flows through precisely one of the separation means. Upstream of the separation means, the outlet stream is divided into one or more portions. Each of the portions is guided through precisely one of the separation means. Downstream of the separation means, the individual portions of the outlet stream may be combined again. It will be appreciated that this applies only to the liquid portions of the outlet stream that are not separated from the outlet stream with the separation means.

The separation unit comprises the separation means. The separation unit thus forms a unit inasmuch as the separation means are subsumed under the generic term separation unit. The separation unit is thus defined as the sum of the separation means. It is not necessary for the separation unit to be a separate component in which all the separation means are integrated. It is sufficient for the separation means to cooperate such that the outlet stream can be divided between them as described and the separation means can thus together perform the function described herein of the separation unit.

The separation unit preferably has exactly two or exactly four separation means.

The outlet stream is divided between the separation means such that the entire outlet stream is guided through one of the separation means or a portion of the outlet stream is guided through each of a plurality of the separation means. Divide thus means that a proportion of the outlet stream is allocated to each of the separation means. For each of the separation means, this proportion can be between 0 and 100%, wherein the sum of the proportions of all the separation means is 100%. Dividing the outlet stream between the separation means thus also includes the limiting case in which a proportion of 0% is allocated to one or more of the separation means, so that the outlet stream does not flow through these separation means at all. Dividing the outlet stream between the separation means also includes the limiting case in which a proportion of 100% is allocated to one of the separation means. In this case, the proportion for all the other separation means must be 0%. That the division also includes the limiting cases is made clear by the fact that it is stated not merely that the outlet stream is divided but that the outlet stream is divided between the separation means such that the entire outlet stream is guided through one of the separation means or a portion of the outlet stream is guided through each of a plurality of the separation means.

The division of the outlet stream between the separation means is adjusted in dependence on a volumetric flow rate of the outlet stream. The described process is thus able to react particularly well to fluctuations in the outlet stream. In particular, it can be achieved that fluctuations of the flow velocity in the individual separation means are comparatively low. The following example illustrates this. The separation unit here has three identical separation means. In the case of a low volumetric flow rate of the outlet stream, the entire outlet stream is guided through only one of the three separation means-which one this is immaterial. It is assumed that the volumetric flow rate of the outlet stream doubles. If a second of the separation means is then added, the flow velocity in the two active separation means is as great as previously in the single active separation means. It is assumed that the volumetric flow rate of the outlet stream increases to three times the starting value. If the third of the separation means is then also added, the flow velocity in all three separation means is as great as previously in the single or in the two active separation means.

The division of the outlet stream between the separation means on the one hand comprises the question of between how many of the separation means the outlet stream is divided. These separation means can be referred to as active separation means. On the other hand, however, it also comprises the question of how the outlet stream is divided between the active separation means. It is preferred, for example, that the number of active separation means is chosen in dependence on the volumetric flow rate of the outlet stream and that the outlet stream is divided in equal portions between the active separation means. Alternatively, it is preferred that the number of active separation means is chosen in dependence on the volumetric flow rate of the outlet stream and that not more than one of the active separation means is operated with only a portion of the maximum capacity of that separation means. A further separation means is thus added whenever the separation means that are already active are being used to full capacity. It is, however, also possible that the number of active separation means is chosen in dependence on the volumetric flow rate of the outlet stream and that it is permitted that the outlet stream distributes itself freely between the active separation means. Division between the active separation means that adjusts itself is thus accepted.

The division of the outlet stream between the separation means is preferably adjusted in dependence on the volumetric flow rate of the outlet stream such that a flow velocity of the waste gas in each of the separation means lies within a predefined range. By dividing the outlet stream between one or more of the separation means, the predefined range for the individual separation means can be comparatively small, in particular compared to the range of flow velocities to which a conventional separator with only a single separation means would be subject in a comparable situation.

The separation means each have a separation capacity. This can be given by a flow cross section, for example. The division of the outlet stream between the separation means is preferably adjusted in dependence on the volumetric flow rate of the outlet stream such that the greater the volumetric flow rate of the outlet stream, the greater a sum of the separation capacities of the active separation means, and vice versa. In particular, it is preferred that the division of the outlet stream between the separation means is adjusted in dependence on the volumetric flow rate of the outlet stream such that the greater the volumetric flow rate of the outlet stream, the greater a sum of the flow cross sections of the active separation means, and vice versa.

The separation means each have a working range. The working range can be defined in particular as a range of the volumetric flow rates which can be applied to the separation means in question. The division of the outlet stream between the separation means is preferably adjusted in dependence on the volumetric flow rate of the outlet stream such that the volumetric flow rate of the outlet stream falls within a working range of the separation unit that is composed of the working ranges of the active separation means.

That the outlet stream is divided between the separation means such that the entire outlet stream is guided through one of the separation means or a portion of the outlet stream is guided through each of a plurality of the separation means, wherein a division of the outlet stream between the separation means is adjusted in dependence on a volumetric flow rate of the outlet stream, can also be expressed in that the entire outlet stream is guided through one of the separation means or a portion of the outlet stream is guided through each of a plurality of the separation means, wherein the size of the proportions of the outlet stream that are guided through each of the individual separation means is adjusted in dependence on a volumetric flow rate of the outlet stream. The proportions can in each case be between 0 and 100%.

The separation unit preferably has a pre-separator and a main separator. In this respect, there are various possibilities. The main separator can have the separation means. A common pre-separator can be arranged upstream of the main separator so composed. Alternatively, the pre-separator can also be subdivided. A plurality of pre-separation means can be arranged upstream of a main separator composed of a plurality of separation means. Alternatively, the separation means can each have a main separation element and a pre-separation element. The main separation elements of the separation means in this case correspond functionally in their totality to a main separator, and the pre-separation elements of the separation means in this case correspond functionally in their totality to a pre-separator. Generally, it is not important for the mode of operation of the described process whether the separator is subdivided into the separation means in relation to the main separating function and/or in relation to the pre-separating function. Preferably, the main separator is in any case divided between a plurality of separation means. The separation means can in particular be configured as compartments of the main separator.

In a preferred embodiment of the process, the division of the outlet stream between the separation means in dependence on the volumetric flow rate of the outlet stream is adjusted in that a number of separation means through which in each case at least a portion of the outlet stream is guided is adjusted.

The outlet stream is divided between the separation means such that the entire outlet stream is guided through one of the separation means or a portion of the outlet stream is guided through each of a plurality of the separation means. Preferably, the greater the volumetric flow rate of the outlet stream, the greater the number of active separation means. In the case of a small volumetric flow rate, it is therefore preferred that the entire outlet stream is guided through a single one of the separation means. As the volumetric flow rate increases, further of the separation means can then gradually be added.

In a further preferred embodiment of the process, the division of the outlet stream between the separation means in dependence on the volumetric flow rate of the outlet stream is adjusted in that a proportion of the outlet stream that is guided through each separation means is adjusted for the separation means.

The proportion can be between 0 and 100% for each of the separation means. Adjustment of the proportion thus also comprises deciding whether a separation means is active or inactive.

In a further preferred embodiment of the process, the outlet stream in a first case has a smaller volumetric flow rate than in a second case, wherein the outlet stream in the first case is guided at least in part through fewer of the separation means than in the second case.

Generally, it is preferred that the greater the volumetric flow rate of the outlet stream, the greater the number of separation means that are active. This dependency is of course not linear but step-like.

In a further preferred embodiment of the process, the outlet stream in a first case has a smaller volumetric flow rate than in a second case, wherein a first of the separation means has a smaller flow cross section than a second of the separation means, and wherein the outlet stream is guided in the first case wholly through the first separation means and in the second case wholly through the second separation means.

In the present embodiment, the separation means are of different sizes. This is expressed by the fact that the first separation means has a smaller flow cross section than the second separation means. In the case of a smaller volumetric flow rate of the outlet stream, the first separation means is therefore used. In the case of a greater volumetric flow rate of the outlet stream, the larger second separation means is used. It is possible to change from the first separation means to the second separation means, for example, when the volumetric flow rate of the outlet stream exceeds a limit value.

In a further preferred embodiment of the process, the outlet stream in a third case has a greater volumetric flow rate than in the second case, wherein the outlet stream in the third case is guided at least in part through the first separation means and through the second separation means. It is possible to change from the second separation means to the combination of the first separation means and the second separation means, for example, when the volumetric flow rate of the outlet stream exceeds a further limit value.

This embodiment builds on the embodiment described above. If the volumetric flow rate of the outlet stream is too great even for the larger second separation means, the smaller first separation means can be connected in addition.

In a further preferred embodiment of the process, the synthesis reactor assembly produces methanol at a production rate of up to 5000 tons of pure methanol per day, particularly preferably of up to 1000 tons per day. This means that 5000 t of methanol molecules are produced.

A further aspect of the invention provides an arrangement for synthesis of methanol, which comprises a synthesis reactor assembly and a separation unit attached to the synthesis reactor assembly, wherein the separation unit is adapted to separate methanol produced with the synthesis reactor assembly from an outlet stream of the synthesis reactor assembly, and wherein the separation unit has a plurality of separation means fluidically connected in parallel.

The described advantages and features of the process are applicable and transferable to the arrangement and vice versa. The arrangement is preferably adapted for operation according to the process. The process is preferably performed with the arrangement.

In a preferred embodiment, the arrangement further has a control unit which is adapted to divide the outlet stream between the separation means such that the entire outlet stream is guided through one of the separation means or a portion of the outlet stream is guided through each of a plurality of the separation means, and to adjust a division of the outlet stream between the separation means in dependence on a volumetric flow rate of the outlet stream.

In this embodiment, the process described above can be performed in an automated manner. The control unit is preferably adapted to perform the described process.

However, it is not essential to use a control unit configured as described. In an alternative, the arrangement can also be used in accordance with the described process without a control unit, for example in that the division of the outlet stream between the separation means is carried out manually. In order to achieve the advantages described herein, it is not necessary that the division takes place in an automated manner. It is sufficient that the arrangement makes it possible for the outlet stream to be divided. This is the case when, in contrast to a conventional separator, the separation unit, as described, has a plurality of separation means fluidically connected in parallel.

In a further preferred embodiment of the arrangement, a flow cross section of each of the separation means is equal in size.

In this embodiment, the division of the outlet stream between the separation means in dependence on the volumetric flow rate of the outlet stream can be adjusted in particular in that the number of active separation means is chosen in dependence on the volumetric flow rate of the outlet stream. This embodiment is particularly simple to implement because, with the same size of separation means, a degree of freedom in the division of the outlet stream is eliminated.

In a further preferred embodiment of the arrangement, a first of the separation means has a smaller flow cross section than a second of the separation means.

In this embodiment, the division of the outlet stream between the separation means in dependence on the volumetric flow rate of the outlet stream can be adjusted in particular in that the number of active separation means is chosen in dependence on the volumetric flow rate of the outlet stream and/or in that it is chosen which of the separation means are active in dependence on the volumetric flow rate. For example, larger separation means can be preferred in the case of a greater volumetric flow rate. This embodiment is more flexible compared to the embodiment described above.

In a further preferred embodiment of the arrangement, the separation means are adapted for the same separation principle.

In this embodiment, the division of the outlet stream between the separation means in dependence on the volumetric flow rate of the outlet stream can be adjusted in particular in that the number of active separation means is chosen in dependence on the volumetric flow rate of the outlet stream. This embodiment is particularly simple to implement because, with the same separation principle, a degree of freedom in the division of the outlet stream is eliminated.

In a further preferred embodiment of the arrangement, a first of the separation means is adapted for a different separation principle than a second of the separation means.

In this embodiment, the division of the outlet stream between the separation means in dependence on the volumetric flow rate of the outlet stream can be adjusted in particular in that the number of active separation means is chosen in dependence on the volumetric flow rate of the outlet stream and/or in that it is chosen which of the separation means are active in dependence on the volumetric flow rate. For example, in the case of a greater volumetric flow rate, preference may be given to the separation means the separation principle of which is suitable for a greater volumetric flow rate. This embodiment is more flexible compared to the embodiment described above.

In a further preferred embodiment of the arrangement, at least some of the separation means are arranged in a common housing.

Particularly preferably, all of the separation means are arranged in the common housing.

Combining the separation means in a common housing is advantageous for practical reasons. In this case, the separation unit is configured as a separate component. This can be incorporated instead of a conventional separator into an arrangement for synthesis of methanol that is otherwise of conventional form. In this respect, the separation unit can also be interpreted as being a separator which has an expanded working range compared to a conventional separator. This is all the more the case when the control unit described herein is integrated into the separation unit or is attached thereto.

In a further preferred embodiment of the arrangement, at least some of the separation means are arranged in different housings.

Particularly preferably, all of the separation means are arranged in different housings. Different combinations with the embodiment described above are, however, also possible.

### BRIEF DESCRIPTION OF THE DRAWINGS

The invention will be explained in greater detail hereinbelow with reference to FIGS. 1 and 4 to 8b. FIGS. 1 and 4 to 8b show particularly preferred embodiments but the invention is not limited thereto. The figures and the size relationships presented therein are only schematic. In the figures:

FIG. 1 shows an inventive arrangement for synthesis of methanol,

FIG. 2a is a perspective view of a separator known from the prior art,

FIG. 2b is a sectional representation from above of the separator known from the prior art of FIG. 2a,

FIG. 3 is a schematic representation of the separator known from the prior art of FIGS. 2a and 2b,

FIG. 4 is a schematic representation of a first embodiment of a separation unit of the inventive arrangement of FIG. 1,

FIG. 5a shows an illustration of a second embodiment of the separation unit of the inventive arrangement of FIG. 1,

FIG. 5b shows an illustration of a second embodiment of the separation unit of the inventive arrangement of FIG. 1,

FIG. 5c shows an illustration of a second embodiment of the separation unit of the inventive arrangement of FIG. 1,

FIG. 6a shows an illustration of a third embodiment of the separation unit of the inventive arrangement of FIG. 1,

FIG. 6b shows an illustration of a third embodiment of the separation unit of the inventive arrangement of FIG. 1,

FIG. 7a shows an illustration of a fourth embodiment of the separation unit of the inventive arrangement of FIG. 1,

FIG. 7b shows an illustration of a fourth embodiment of the separation unit of the inventive arrangement of FIG. 1,

FIG. 8a shows an illustration of a fifth embodiment of the separation unit of the inventive arrangement of FIG. 1, and

FIG. 8b shows an illustration of a fifth embodiment of the separation unit of the inventive arrangement of FIG. 1.

### DETAILED DESCRIPTION OF THE PREFERRED EMBODIMENTS

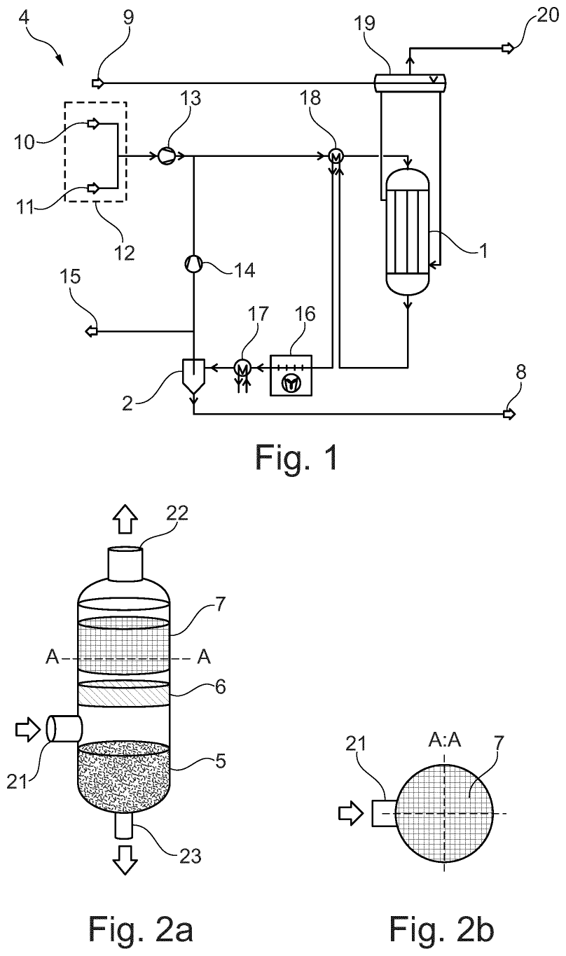

FIG. 1 shows an arrangement 4 for synthesis of methanol. The arrangement 4 comprises a raw gas source 12, a synthesis reactor assembly 1 and a separation unit 2 attached thereto. The separation unit 2 is adapted to separate methanol produced with the synthesis reactor assembly 1 from an outlet stream of the synthesis reactor assembly 1. The separated methanol can be discharged at a methanol outlet 8. The raw gas source 12 comprises an H₂ source 11 and a CO₂ source 10. The raw gas source 12, the synthesis reactor assembly 1 and the separation unit 2 are connected to one another via a pipe conduit system. The arrangement 4 further comprises a water feed conduit 9 which is connected with a steam drum 19. Steam from the steam drum 19 can be discharged via a steam outlet 20.

Further integrated into the pipe conduit system are a first cooler 16, a second cooler 17, a heat exchanger 18, a first compressor 13 and a second compressor 14. In the event of overpressure in the pipe conduit system gas may be discharged via an overpressure outlet 15.

FIGS. 2a and 2b show a separator known from the prior art. This could be used instead of the separation unit 2 in an arrangement analogous to that of FIG. 1. The separator has an inlet 21, a gas outlet 22 and a liquid outlet 23. A pre-separator 6 and a main separator 7 are arranged in a housing 5 of the separator. If an outlet stream of a synthesis reactor assembly enters the separator via the inlet 21, liquid and gas in the outlet stream are separated by the pre-separator 6 and the main separator 7. The liquid collects in the lower region of the housing 5 and can be discharged via the liquid outlet 23. The gas rises in the housing 5 and can be discharged via the gas outlet 22. In the known separator it is possible only to guide the entire outlet stream through the separator. This is disadvantageous in the case of a fluctuating outlet stream.

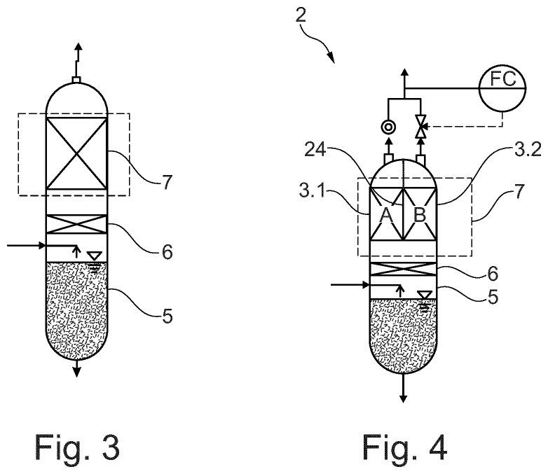

FIG. 3 is a schematic representation of the separator known from the prior art of FIGS. 2a and 2b.

FIG. 4, analogously to FIG. 3, is a schematic representation of a first embodiment of a separation unit 2 of the inventive arrangement 4 of FIG. 1. The separation unit 2 also has a housing 5 in which a pre-separator 6 and a main separator 7 are formed. However, the main separator 7 is in this case formed by a first separation means 3.1 and a second separation means 3.2, which are separated from one another by a partition wall 24.

The separation means 3.1, 3.2 are fluidically connected in parallel. As a result, the outlet stream of the synthesis reactor assembly 1 can be divided between the separation means 3.1, 3.2 such that the entire outlet stream is guided through one of the separation means 3.1, 3.2 or a portion of the outlet stream is guided through each of the two separation means 3.1, 3.2, wherein a division of the outlet stream between the separation means 3.1, 3.2 is adjusted in dependence on a volumetric flow rate of the outlet stream.

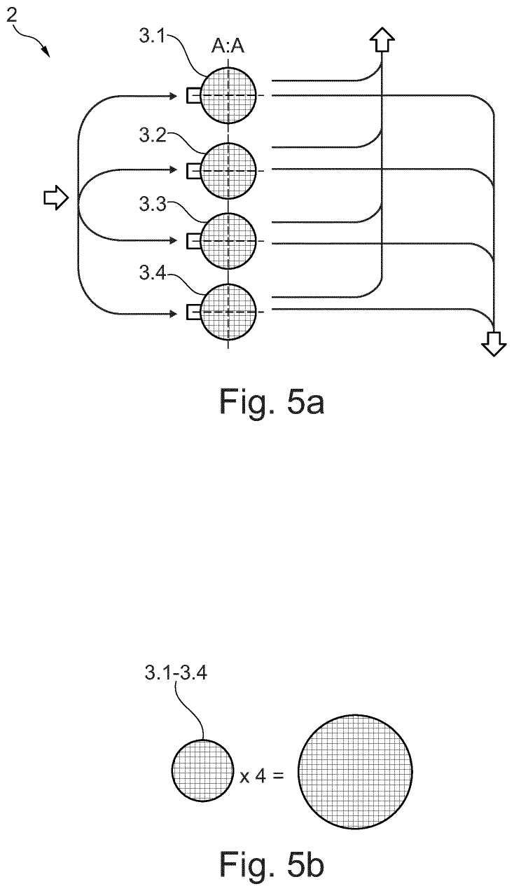

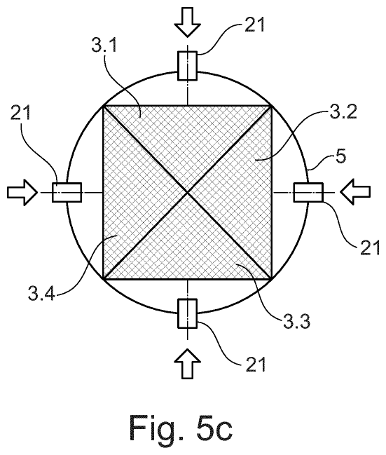

FIG. 5a shows an illustration of a second embodiment of the separation unit 2 of the inventive arrangement 4 of FIG. 1. The separation unit 2 has four separation means 3.1 to 3.4 fluidically connected in parallel. The separation means 3.1 to 3.4 are identical in form. In particular, a flow cross section of the separation means 3.1 to 3.4 is thus in each case equal in size and the separation means 3.1 to 3.4 are adapted for the same separation principle.

The division of the outlet stream between the separation means 3.1 to 3.4 in dependence on the volumetric flow rate of the outlet stream can be adjusted in this embodiment in that a proportion of the outlet stream that is guided through the respective separation means 3.1 to 3.4 is adjusted for each of the separation means 3.1 to 3.4. In particular, a number of separation means 3.1 to 3.4 through each of which at least a portion of the outlet stream is guided can be adjusted. If the outlet stream in a first case has a smaller volumetric flow rate than in a second case, the outlet stream can in the first case be guided at least in part through fewer of the separation means 3.1 to 3.4 than in the second case.

The separation unit 2 of FIG. 5a can be operated, for example, as follows:
at 15 to 30% of a reference volumetric flow rate of the outlet stream, one of the separation means 3.1 to 3.4 is active,
at 30 to 45% of a reference volumetric flow rate of the outlet stream, two of the separation means 3.1 to 3.4 are active,
at 45 to 60% of a reference volumetric flow rate of the outlet stream, two or three of the separation means 3.1 to 3.4 are active-this can be decided on the basis of a further decision criterion,
at 60 to 90% of a reference volumetric flow rate of the outlet stream, three or four of the separation means 3.1 to 3.4 are active-this can be decided on the basis of a further decision criterion,
at 90 to 120% of a reference volumetric flow rate of the outlet stream, all four of the separation means 3.1 to 3.4 are active.

The reference volumetric flow rate can be defined on the basis of a nominal capacity, which can be exceeded by up to 120% thereof.

FIG. 5b illustrates that the flow cross section of a single one of the separation means 3.1 to 3.4, when multiplied by a factor of four, corresponds to a large overall flow cross section. If all four separation means 3.1 to 3.4 are active, the separation unit 2 has the same function as a simple separator with a flow cross section of the size shown on the right in FIG. 5b.

FIG. 5c shows, in a sectional representation from above, how the embodiment of FIGS. 5a and 5b can be implemented by way of example in practice. The four separation means 3.1 to 3.4 are arranged in a common housing 5. Each of the separation means 3.1 to 3.4 has a respective inlet 21, so that flow can take place at the separation means 3.1 to 3.4 individually.

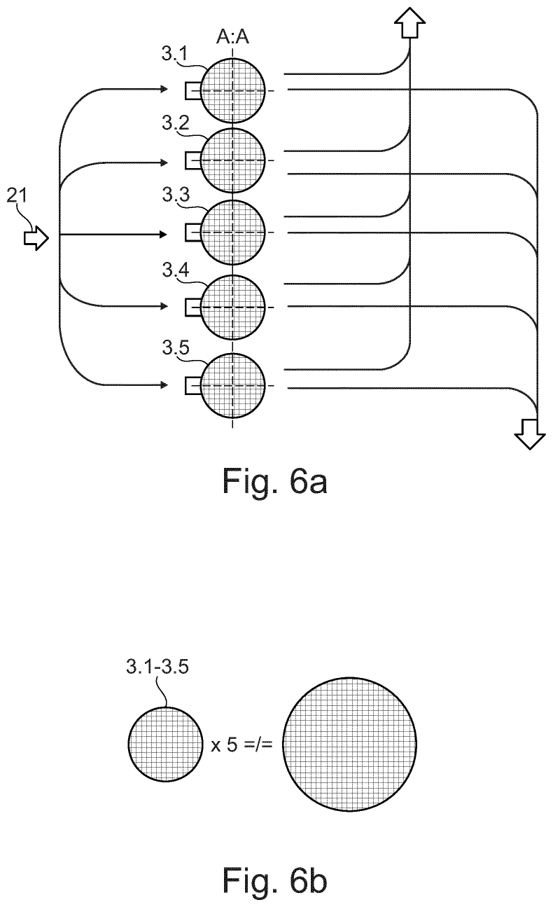

FIGS. 6a and 6b, analogously to FIGS. 5a and 5b, show a third embodiment of the separation unit 2 of the inventive arrangement 4 of FIG. 1. In the case of FIGS. 6a and 6b, however, a fifth separation means 3.5 is additionally provided.

The separation unit 2 of FIGS. 6a and 6b can be operated, for example, as follows:
at 15 to 30% of a reference volumetric flow rate of the outlet stream, one of the separation means 3.1 to 3.4 is active,
at 30 to 45% of a reference volumetric flow rate of the outlet stream, two of the separation means 3.1 to 3.4 are active,
at 45 to 60% of a reference volumetric flow rate of the outlet stream, two or three of the separation means 3.1 to 3.4 are active-this can be decided on the basis of a further decision criterion,
at 60 to 75% of a reference volumetric flow rate of the outlet stream, three or four of the separation means 3.1 to 3.4 are active-this can be decided on the basis of a further decision criterion,
at 75 to 120% of a reference volumetric flow rate of the outlet stream, four or five of the separation means 3.1 to 3.5 are active-this can be decided on the basis of a further decision criterion,
at 120 to 150% of a reference volumetric flow rate of the outlet stream, all five of the separation means 3.1 to 3.5 are active.

In this case it is assumed that the reference volumetric flow rate can be exceeded by up to 150% thereof.

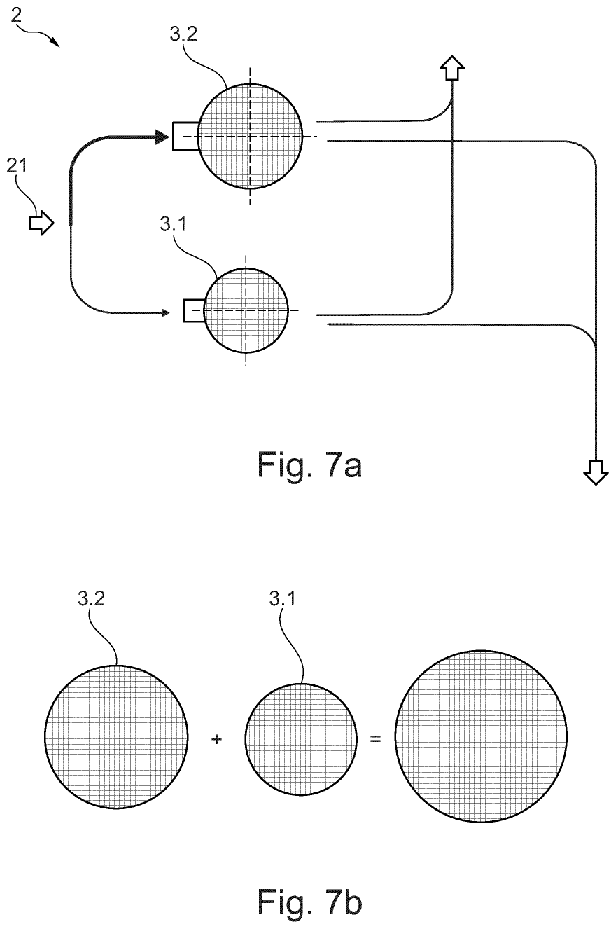

FIGS. 7a and 7b illustrate a fourth embodiment of the separation unit 2 of the inventive arrangement 4 of FIG. 1. In this case, the first separation means 3.1 has a smaller flow cross section than the second separation means 3.2. It is assumed that the outlet stream in a first case has a smaller volumetric flow rate than in a second case and a higher volumetric flow rate in a third case than in the second case. The outlet stream can then be guided in the first case wholly through the first separation means 3.1, in the second case wholly through the second separation means 3.2, and in the third case in part through each of the first separation means 3.1 and the second separation means 3.2. The third case is illustrated in FIG. 7 b.

The separation unit 2 of FIGS. 7a and 7b can be operated, for example, as follows:
at 20 to 40% of a reference volumetric flow rate of the outlet stream, only the first separation means 3.1 is active,
at 40 to 60% of a reference volumetric flow rate of the outlet stream, only the second separation means 3.2 is active,
at 60 to 80% of a reference volumetric flow rate of the outlet stream, only the second separation means 3.2 is active or both separation means 3.1, 3.2 are active-this can be decided on the basis of a further decision criterion,
at 80 to 120% of a reference volumetric flow rate of the outlet stream, both separation means 3.1, 3.2 are active.

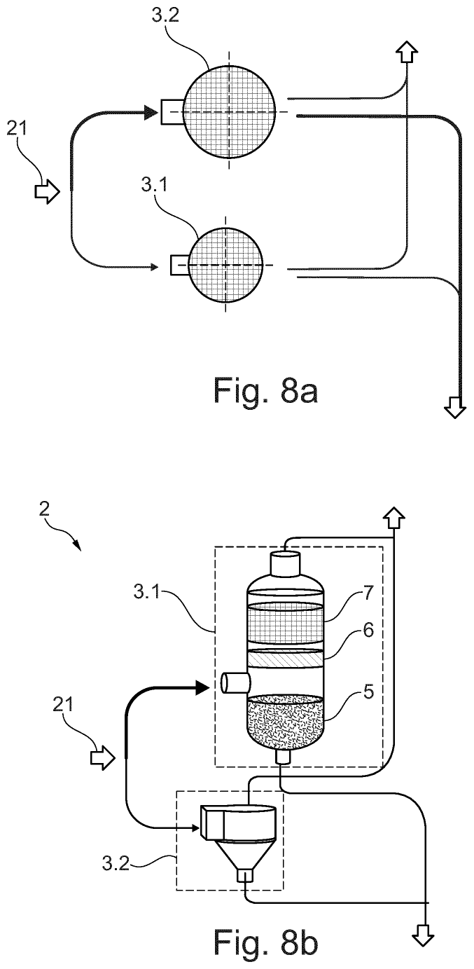

FIGS. 8a and 8b illustrate a fifth embodiment of the separation unit 2 of the inventive arrangement 4 of FIG. 1. In this case, the first separation means 3.1 is adapted for a different separation principle than the second separation means 3.2. In this embodiment, the outlet stream can be divided analogously to the fourth embodiment according to FIGS. 7a and 7b.

FIG. 8b shows a possibility for implementing the fifth embodiment. The first separation means 3.1 is here configured like the separator known from the prior art of FIGS. 2a and 2 b. However, the second separation means 3.2 is for this purpose connected in parallel. In this case, the first separation means 3.1 is adapted for a different separation principle than the second separation means 3.2. Although the first separation means 3.1 and the second separation means 3.2 are formed in different housings, they nevertheless together form the separation unit 2.

### LIST OF REFERENCE NUMERALS

1 Synthesis reactor assembly
2 Separation unit
3.1 First separation means
3.2 Second separation means
3.3 Third separation means
3.4 Fourth separation means
3.5 Fifth separation means
4 Arrangement
5 Housing
6 Pre-separator
7 Main separator
8 Methanol outlet
9 Water feed conduit
10 CO₂ source
11 H₂ source
12 Raw gas source
13 First compressor
14 Second compressor
15 Overpressure outlet
16 First cooler
17 Second cooler
18 Heat exchanger
19 Steam drum
20 Steam outlet
21 Inlet
22 Gas outlet
23 Liquid outlet
24 Partition wall

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

1.-14. (canceled)

15. A process for synthesis of methanol with a synthesis reactor assembly, comprising:
Separating the methanol produced with the synthesis reactor assembly with a separation unit from an outlet stream of the synthesis reactor assembly,
connecting the separation unit, comprising a plurality of separation means, fluidically in parallel, ·dividing the outlet stream between the separation means such that the entire outlet stream is guided through one of the separation means, or a portion of the outlet stream is guided through each of a plurality of the separation means, and
adjusting a division of the outlet stream between the separation means in dependence on a volumetric flow rate of the outlet stream.

16. The process according to claim 15, wherein the division of the outlet stream between the separation means in dependence on the volumetric flow rate of the outlet stream is adjusted in that a number of separation means through which in each case at least a portion of the outlet stream is guided is adjusted.

17. The process according to claim 15, wherein the division of the outlet stream between the separation means in dependence on the volumetric flow rate of the outlet stream is adjusted in that a proportion of the outlet stream that is guided through each separation means is adjusted for the separation means.

18. The process according to claim 15, wherein the outlet stream in a first case has a smaller volumetric flow rate than in a second case, and wherein the outlet stream in the first case is guided at least in part through fewer of the separation means than in the second case.

19. The process according to claim 15, wherein the outlet stream in a first case has a smaller volumetric flow rate than in a second case, wherein a first of the separation means has a smaller flow cross section than a second of the separation means, and wherein the outlet stream is guided in the first case wholly through the first separation means and in the second case wholly through the second separation means.

20. The process according to claim 19, wherein the outlet stream in a third case has a greater volumetric flow rate than in the second case, and wherein the outlet stream in the third case is guided in part through the first separation means and through the second separation means.

21. An arrangement for synthesis of methanol, comprising
a synthesis reactor assembly and a separation unit attached to the synthesis reactor assembly,
wherein the separation unit is adapted to separate methanol produced with the synthesis reactor assembly from an outlet stream of the synthesis reactor assembly and
wherein the separation unit has a plurality of separation means fluidically connected in parallel.

22. The arrangement according to claim 21, further comprising a control unit which is adapted to divide the outlet stream between the separation means such that the entire outlet stream is guided through one of the separation means or a portion of the outlet stream is guided through each of a plurality of the separation means, and to adjust a division of the outlet stream between the separation means in dependence on a volumetric flow rate of the outlet stream.

23. The arrangement according to claim 21, wherein a flow cross section of each of the separation means is equal in size.

24. The arrangement according to claim 21, wherein a first of the separation means has a smaller flow cross section than a second of the separation means.

25. The arrangement according to claim 21, wherein the separation means are adapted for the same separation principle.

26. The arrangement according claim 21, wherein a first of the separation means is adapted for a different separation principle than a second of the separation means.

27. The arrangement according to claim 21, wherein at least some of the separation means are arranged in a common housing.

28. The arrangement according to claim 21, wherein at least some of the separation means are arranged in different housings.
